SEQUENCE LISTING (4 sequences)

SEQ ID NO: 1 - DNA, 31 bp, Artificial sequence
  Primer (1)..(31): eno1
gtttgtctagagtttcagtttaactagtgac

SEQ ID NO: 2 - DNA, 25 bp, Artificial sequence
  Primer (1)..(25): eno2
ccggaggctggcaagcttaaatcag

SEQ ID NO: 3 - DNA, 1381 bp, Escherichia coli
  PCR product (1)..(1381)
gtttgtctagagtttcagtttaactagtgacttgaggaaaacctaatgtccaaaatcgtaaaaatcatcggtcgtgaaatcatcgactcccgtggtaacccgactgttgaagccgaagtacatctggagggtggtttcgtcggtatggcagctgctccgtcaggtgcttctactggttcccgtgaagctctggaactgcgcgatggcgacaaatcccgtttcctgggtaaaggcgtaaccaaagctgttgctgcggtaaacggcccgatcgctcaggcgctgattggcaaagatgctaaagatcaggctggcattgacaagatcatgatcgacctggacggcaccgaaaacaaatccaaattcggcgcgaacgcaatcctggctgtatctctggctaacgccaaagctgctgcagctgctaaaggtatgccgctgtacgagcacatcgctgaactgaacggtactccgggcaaatactctatgccggttccgatgatgaacatcatcaacggtggtgagcacgctgacaacaacgttgatatccaggaattcatgattcagccggttggcgcgaaaactgtgaaagaagccatccgcatgggttctgaagttttccatcacctggcaaaagttctgaaagcgaaaggcatgaacactgctgttggtgacgaaggtggctatgcgccgaacctgggttccaacgctgaagctctggctgttatcgctgaagctgttaaagctgctggttatgaactgggcaaagacatcactttggcgatggactgcgcagcttctgaattctacaaagatggtaaatacgttctggctggcgaaggcaacaaagcgttcacctctgaagaattcactcacttcctggaagaactgaccaaacagtacccgatcgtttctatcgaagacggtctggacgaatctgactgggacggtttcgcataccagaccaaagttctgggcgacaaaatccagctggttggtgacgacctgttcgtaaccaacaccaagatcctgaaagaaggtatcgaaaaaggtatcgctaactccatcctgatcaaattcaaccagatcggttctctgaccgaaactctggctgcaatcaagatggcgaaagatgctggctacactgcagttatctctcaccgttctggcgaaactgaagacgctaccatcgctgacctggctgttggtactgctgcaggccagatcaaaactggttctatgagccgttctgaccgtgttgctaaatacaaccagctgattcgtatcgaagaagctctgggcgaaaaagcaccgtacaacggtcgtaaagagatcaaaggccaggcataagactgactttatctgatttaagcttgccagcctccgg

SEQ ID NO: 4 - protein, 432 aa, Escherichia coli
MSKIVKIIGREIIDSRGNPTVEAEVHLEGGFVGMAAAPSGASTGSREALELRDGDKSRFLGKGVTKAVAAVNGPIAQALIGKDAKDQAGIDKIMIDLDGTENKSKFGANAILAVSLANAKAAAAAKGMPLYEHIAELNGTPGKYSMPVPMMNIINGGEHADNNVDIQEFMIQPVGAKTVKEAIRMGSEVFHHLAKVLKAKGMNTAVGDEGGYAPNLGSNAEALAVIAEAVKAAGYELGKDITLAMDCAASEFYKDGKYVLAGEGNKAFTSEEFTHFLEELTKQYPIVSIEDGLDESDWDGFAYQTKVLGDKIQLVGDDLFVTNTKILKEGIEKGIANSILIKFNQIGSLTETLAAIKMAKDAGYTAVISHRSGETEDATIADLAVGTAAGQIKTGSMSRSDRVAKYNQLIRIEEALGEKAPYNGRKEIKGQA

(19) United States
(12) Patent Application Publication
(10) Pub. No.: US 2005/0164357 A1
(43) Pub. Date: Jul. 28, 2005

(54) Process for the preparation of L-amino acids using strains of the Enterobacteriaceae family

(76) Inventor: Mechthild Rieping, Bielefeld (DE)

Correspondence Address: Michael A. Sanzo;Fitch, Even, Tabin & Flannery, 1801 K. Street. N.W., Suite 401L, Washington, DC, 20006-1201, US

(21) Appl. No.: 10/784,914

(22) Filed: Feb. 24, 2004

(30) Foreign Application Priority Data
Jan. 23, 2004 (DE) 10 2004 003 410.9

(51) Int. Cl. C12P 13/04; C12N 1/21

(52) U.S. Cl. 435/106; 435/252.33

(57) ABSTRACT

The invention relates to a process for the preparation of L-amino acids, in particular L-threonine, in which the following steps are carried out: a) fermentation of the microorganisms of the Enterobacteriaceae family which produce the desired L-amino acid and in which the eno gene or nucleotide sequences or alleles which code for it are enhanced, b) concentration of the desired L-amino acid in the medium or in the cells of the microorganisms, and c) isolation of the desired L-amino acid.

### FIELD OF THE INVENTION

This invention relates to a process for the preparation of L-amino acids, in particular L-threonine, using strains of the Enterobacteriaceae family in which the eno gene is enhanced.

### BACKGROUND OF THE INVENTION

L-Amino acids, in particular L-threonine, are used in human medicine and in the pharmaceuticals industry, in the foodstuffs industry and very particularly in animal nutrition.

It is known that L-amino acids are prepared by fermentation of strains of Enterobacteriaceae, in particular Escherichia coli (E. coli) and Serratia marcescens. Because of their great importance, work is constantly being undertaken to improve the preparation processes. Improvements to the process can relate to fermentation measures, such as e.g. stirring and supply of oxygen, or the composition of the nutrient media, such as e.g. the sugar concentration during the fermentation, or the working up to the product form, by e.g. ion exchange chromatography, or the intrinsic output properties of the microorganism itself.

Methods of mutagenesis, selection and mutant selection are used to improve the output properties of these microorganisms. Strains which are resistant to antimetabolites, such as e.g. the threonine analogue α-amino-β-hydroxyvaleric acid (AHV), or are auxotrophic for metabolites of regulatory importance and produce L-amino acid, such as e.g. L-threonine, are obtained in this manner.

Methods of the recombinant DNA technique have also been employed for some years for improving the strain of strains of the Enterobacteriaceae family which produce L-amino acids, by amplifying individual amino acid biosynthesis genes and investigating the effect on the production. Summarizing information on the cell and molecular biology of Escherichia coli and Salmonella are to be found in Neidhardt (ed): Escherichia coli and Salmonella, Cellular and Molecular Biology, 2nd Edition, ASM Press, Washington, D.C., USA (1996).

### OBJECT OF THE INVENTION

The object of the invention is to provide new measures for improved fermentative preparation of L-amino acids, in particular L-threonine.

### SUMMARY OF THE INVENTION

The invention provides a process for the preparation of L-amino acids, in particular L-threonine, using microorganisms of the Enterobacteriaceae family which, in particular, already produce L-amino acids and in which at least the nucleotide sequence which codes for the eno gene or alleles thereof is or are enhanced.

### DETAILED DESCRIPTION OF THE INVENTION

Where L-amino acids or amino acids are mentioned in the following, this means one or more amino acids, including their salts, chosen from the group consisting of L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan and L-arginine. L-Threonine is particularly preferred.

The term “enhancement” in this connection describes the increase in the intracellular activity or concentration of one or more enzymes or proteins in a microorganism which are coded by the corresponding DNA, for example by increasing the number of copies of the gene or genes by at least one (1) copy, using a potent promoter or a gene or allele which codes for a corresponding enzyme or protein with a high activity, and optionally combining these measures.

By enhancement measures the activity or concentration of the corresponding protein is in general increased by at least 10%, 25%, 50%, 75%, 100%, 150%, 200%, 300%, 400% or 500%, up to a maximum of 1,000% or 2,000%, based on that of the wild-type protein or the activity or concentration of the protein in the starting microorganism. The starting microorganism is understood as meaning the microorganism on which the inventive measures are carried out.

The process according to the invention is characterized in that the following steps are carried out:
a) fermentation of the microorganisms of the Enterobacteriaceae family which produce the desired L-amino acid and in which the eno gene or nucleotide sequences or alleles which code for it are enhanced, in a medium under conditions suitable for the formation of the eno gene product,
b) concentration of the desired L-amino acid in the medium or in the cells of the microorganisms, and
c) isolation of the desired L-amino acid, constituents of the fermentation broth and/or the biomass in its entirety or portions (≧0 to 100%) thereof optionally remaining in the product or being completely removed.

The microorganisms, in particular recombinant microorganisms, which the present invention provides can produce L-amino acids from glucose, sucrose, lactose, fructose, maltose, molasses, optionally starch, optionally cellulose or from glycerol and ethanol. They are representatives of the Enterobacteriaceae family chosen from the genera Escherichia, Erwinia, Providencia and Serratia. The genera Escherichia and Serratia are preferred. Of the genus Escherichia in particular the species Escherichia coli and of the genus Serratia in particular the species Serratia marcescens are to be mentioned.

Recombinant microorganisms are produced by transformation, conjugation or transduction with vectors carrying the desired genes.

Suitable strains, which produce L-threonine in particular, of the genus Escherichia, in particular of the species Escherichia coli, are, for example

| | |
|---|---|
| Escherichia coli H4581 | (EP 0 301 572) |
| Escherichia coli KY10935 | (Bioscience |
| | Biotechnology and |
| | Biochemistry 61(11): |
| | 1877-1882 (1997) |
| Escherichia coli VNIIgenetika MG442 | (US-A-4278,765) |
| Escherichia coli VNIIgenetika M1 | (US-A-4,321,325) |
| Escherichia coli VNIIgenetika 472T23 | (US-A-5,631,157) |
| Escherichia coli BKIIM B-3996 | (US-A-5,175,107) |
| Escherichia coli kat 13 | (WO 98/04715) |
| Escherichia coli KCCM-10132 | (WO 00/09660). |

Suitable L-threonine-producing strains of the genus Serratia, in particular of the species Serratia marcescens, are, for example
Serratia marcescens HNr21 (Applied and Environmental Microbiology 38 (6): 1045-1051 (1979))
Serratia marcescens TLr156 (Gene 57 (2-3): 151-158 (1987))
Serratia marcescens T-2000 (Applied Biochemistry and Biotechnology 37 (3): 255-265 (1992)).

Strains from the Enterobacteriaceae family which produce L-threonine preferably have, inter alia, one or more genetic or phenotypic features chosen from the group consisting of: resistance to α-amino-β-hydroxyvaleric acid, resistance to thialysine, resistance to ethionine, resistance to α-methylserine, resistance to diaminosuccinic acid, resistance to α-aminobutyric acid, resistance to borrelidin, resistance to cyclopentane-carboxylic acid, resistance to rifampicin, resistance to valine analogues, such as, for example, valine hydroxamate, resistance to purine analogues, such as, for example, 6-dimethylaminopurine, a need for L-methionine, optionally a partial and compensable need for L-isoleucine, a need for meso-diaminopimelic acid, auxotrophy in respect of threonine-containing dipeptides, resistance to L-threonine, resistance to threonine raffinate, resistance to L-homoserine, resistance to L-lysine, resistance to L-methionine, resistance to L-glutamic acid, resistance to L-aspartate, resistance to L-leucine, resistance to L-phenylalanine, resistance to L-serine, resistance to L-cysteine, resistance to L-valine, sensitivity to fluoropyruvate, defective threonine dehydrogenase, optionally an ability for sucrose utilization, enhancement of the threonine operon, enhancement of homoserine dehydrogenase I-aspartate kinase I, preferably of the feed back resistant form, enhancement of homoserine kinase, enhancement of threonine synthase, enhancement of aspartate kinase, optionally of the feed back resistant form, enhancement of aspartate semialdehyde dehydrogenase, enhancement of phosphoenol pyruvate carboxylase, optionally of the feed back resistant form, enhancement of phosphoenol pyruvate synthase, enhancement of transhydrogenase, enhancement of the RhtB gene product, enhancement of the RhtC gene product, enhancement of the YfiK gene product, enhancement of a pyruvate carboxylase, and attenuation of acetic acid formation.

It has been found that microorganisms of the Enterobacteriaceae family produce L-amino acids, in particular L-threonine, in an improved manner after enhancement, in particular over-expression, of the eno gene.

The nucleotide sequences of the genes of Escherichia coli belong to the prior art (see the following text references) and can also be found in the genome sequence of Escherichia coli published by Blattner et al. (Science 277: 1453-1462 (1997)).

The eno gene is described, inter alia, by the following data:

| | |
|---|---|
| Description: | enolase, phosphopyruvate hydratase |
| Function: | enolization (splitting of water): |
| | 2-phosphoglycerate to phosphoenol pyruvate |
| | in glycolysis |
| EC No.: | EC 4.2.1.11 |
| Reference: | Spring TG. and Wold F.; The Journal of |
| | Biological Chemistry 246(22): 6797-6802 |
| | (1971) |
| | Klein et al.; DNA Sequence 6(6): 351-355 |
| | (1996) |
| | Gulick et al.; Biochemistry 40(51): 15716-15724 |
| | (2001) |
| | Kaga et al.; Bioscience, Biotechnology and |
| | Biochemistry 66(10): 2216-2220 (2002) |
| Accession No.: | AE000361 |

The eno gene from Salmonella typhimurium is described, inter alia, in the following reference: Garrido-Pertierra A.; Revista Espanola de Fisiologia 36 (1): 33-39 (1980).

The nucleic acid sequences can be found in the databanks of the National Center for Biotechnology Information (NCBI) of the National Library of Medicine (Bethesda, Md., USA), the nucleotide sequence databank of the European Molecular Biologies Laboratories (EMBL, Heidelberg, Germany or Cambridge, UK) or the DNA databank of Japan (DDBJ, Mishima, Japan).

For better clarity, the nucleotide sequence of the eno gene and the amino acid sequence of the gene product of Escherichia coli are reproduced as SEQ ID NO:3 and 4.

The genes described in the text references mentioned can be used according to the invention. Alleles of the genes which result from the degeneracy of the genetic code or due to “sense mutations” of neutral function can furthermore be used. The use of endogenous genes is preferred.

“Endogenous genes” or “endogenous nucleotide sequences” are understood as meaning the genes or alleles or nucleotide sequences present in the population of a species.

Suitable alleles of the eno gene include those which contain neutral-function mutations or “sense mutations”. These include, inter alia, those which lead to at least one (1) conservative amino acid exchange in the protein coded by them. The maximum number of conservative amino acid exchanges can relate to 2, 3, 5, 10, 20 but in no case more than 30 amino acids. By the conservative amino acid exchanges mentioned, the functional capacity is lowered or increased by 0% to not more than 24%, 20%, 10%, 5%, 3%, 2% or 1%.

In the case of aromatic amino acids, conservative exchanges are referred to when phenylalanine, tryptophan and tyrosine are exchanged for one another. In the case of hydrophobic amino acids, conservative exchanges are referred to when leucine, isoleucine and valine are exchanged for one another. In the case of polar amino acids, conservative exchanges are referred to when glutamine and asparagine are exchanged for one another. In the case of basic amino acids, conservative exchanges are referred to when arginine, lysine and histidine are exchanged for one another. In the case of acidic amino acids, conservative exchanges are referred to when aspartic acid and glutamic acid are exchanged for one another. In the case of amino acids containing hydroxyl groups, conservative exchanges are referred to when serine and threonine are exchanged for one another. All other amino acid exchanges are called non-conservative amino acid exchanges.

In the same way, those nucleotide sequences which code for variants of the proteins mentioned which additionally contain a lengthening or shortening by at least one (1) amino acid on the N or C terminus can also be used. This lengthening or shortening is not more than 50, 40, 30, 20, 10, 5, 3 or 2 amino acids or amino acid radicals.

Suitable alleles also include those which code for proteins in which at least one (1) amino acid is inserted (insertion) or removed (deletion). The maximum number of such changes, called indels, can relate to 2, 3, 5, 10, 20 but in no case more than 30 amino acids.

Suitable alleles furthermore include those which are obtainable by hybridization, in particular under stringent conditions, using SEQ ID No. 3 or parts thereof, in particular the coding regions or the sequences complementary thereto.

Instructions for identifying DNA sequences by means of hybridization can be found by the expert, inter alia, in the handbook “The DIG System Users Guide for Filter Hybridization” from Boehringer Mannheim GmbH (Mannheim, Germany, 1993) and in Liebl et al. (International Journal of Systematic Bacteriology 41: 255-260 (1991)). The hybridization takes place under stringent conditions, that is to say only hybrids in which the probe and target sequence, i.e. the polynucleotides treated with the probe, are at least 70% identical are formed. It is known that the stringency of the hybridization, including the washing steps, is influenced or determined by varying the buffer composition, the temperature and the salt concentration.

The hybridization reaction is in general carried out under a relatively low stringency compared with the washing steps (Hybaid Hybridisation Guide, Hybaid Limited, Teddington, UK, 1996).

A buffer corresponding to 5×SSC buffer at a temperature of approx. 50° C.-68° C., for example, can be employed for the hybridization reaction. Probes can also hybridize here with polynucleotides which are less than 70% identical to the sequence of the probe. Such hybrids are less stable and are removed by washing under stringent conditions. This can be achieved, for example, by lowering the salt concentration to 2×SSC and optionally subsequently 0.5×SSC (The DIG System User's Guide for Filter Hybridisation, Boehringer Mannheim, Mannheim, Germany, 1995) a temperature of approx. 50° C.-68° C., approx. 52° C.-68° C., approx. 54° C.-68° C., approx. 56° C.-68° C., approx. 58° C.-68° C., approx. 60° C.-68° C., approx. 62-68° C., approx. 64° C.-68° C., approx. 66° C.-68° C. being established. It is optionally possible to lower the salt concentration to a concentration corresponding to 0.2×SSC or 0.1×SSC. Polynucleotide fragments which are, for example, at least 70% or at least 80% or at least 90% to 95% or at least 96% to 99% identical to the sequence of the probe employed can be isolated by increasing the hybridization temperature stepwise from 50° C. to 68° C. in steps of approx. 1-2° C. Further instructions on hybridization are obtainable on the market in the form of so-called kits (e.g. DIG Easy Hyb from Roche Diagnostics GmbH, Mannheim, Germany, Catalogue No. 1603558).

To achieve an enhancement, for example, expression of the genes or the catalytic properties of the enzyme proteins can be increased. The two measures can optionally be combined.

Thus, for example, the number of copies of the corresponding genes can be increased, or the promoter and regulation region or the ribosome binding site upstream of the structural gene can be mutated. Expression cassettes which are incorporated upstream of the structural gene act in the same way. By inducible promoters, it is additionally possible to increase the expression in the course of fermentative L-threonine production. The expression is likewise improved by measures to prolong the life of the m-RNA. Furthermore, the enzyme activity is also increased by preventing the degradation of the enzyme protein. The genes or gene constructs can either be present in plasmids with a varying number of copies, or can be integrated and amplified in the chromosome. Alternatively, an over-expression of the genes in question can furthermore be achieved by changing the composition of the media and the culture procedure.

The expert can find instructions in this respect, inter alia, in Chang and Cohen (Journal of Bacteriology 134: 1141-1156 (1978)), in Hartley and Gregori (Gene 0.13: 347-353 (1981)), in Amann and Brosius (Gene 40: 183-190 (1985)), in de Broer et al. (Proceedings of the National Academy of Sciences of the United States of America 80: 21-25 (1983)), in LaVallie et al. (BIO/TECHNOLOGY 11: 187-193 (1993)), in PCT/US97/13359, in Llosa et al. (Plasmid 26: 222-224 (1991)), in Quandt and Klipp (Gene 80: 161-169 (1989)), in Hamilton et al. (Journal of Bacteriology 171: 4617-4622 (1989)), in Jensen and Hammer (Biotechnology and Bioengineering 58: 191-195 (1998)) and in known textbooks of genetics and molecular biology.

Plasmid vectors which can replicate in Enterobacteriaceae, such as e.g. cloning vectors derived from pACYC184 (Bartolome et al.; Gene 102: 75-78 (1991)), pTrc99A (Amann et al.; Gene 69: 301-315 (1988)) or pSC101 derivatives (Vocke and Bastia; Proceedings of the National Academy of Sciences USA 80 (21): 6557-6561 (1983)) can be used. A strain transformed with a plasmid vector where the plasmid vector carries at least one nucleotide sequence or allele which codes for the eno gene can be employed in a process according to the invention.

The term transformation is understood as meaning the uptake of an isolated nucleic acid by a host (microorganism).

It is also possible to transfer mutations which affect the expression of the particular genes into various strains by sequence exchange (Hamilton et al.; (Journal of Bacteriology 171: 4617-4622 (1989)), conjugation or transduction.

More detailed explanations of the terms in genetics and molecular biology are found in known textbooks of genetics and molecular biology, such as, for example, the textbook by Birge (Bacterial and Bacteriophage Genetics, 4th ed., Springer Verlag, New York (USA), 2000) or the textbook by Berg, Tymoczko and Stryer (Biochemistry, 5th ed., Freeman and Company, New York (USA), 2002) or the handbook by Sambrook et al. (Molecular Cloning, A Laboratory Manual, (3 volume set), Cold Spring Harbor Laboratory Press, Cold Spring Harbor (USA), 2001).

It may furthermore be advantageous for the production of L-amino acids, in particular L-threonine, with strains of the Enterobacteriaceae family to enhance one or more enzymes of the known threonine biosynthesis pathway or enzymes of anaplerotic metabolism or enzymes for the production of reduced nicotinamide adenine dinucleotide phosphate or enzymes of glycolysis or PTS enzymes or enzymes of sulfur metabolism, in addition to the enhancement of the eno gene. The use of endogenous genes is in general preferred.

Thus, for example, at the same time one or more of the genes chosen from the group consisting of
the thrABC operon which codes for aspartate kinase, homoserine dehydrogenase, homoserine kinase and threonine synthase (U.S. Pat. No. 4,278,765),
the pyc gene of Corynebacterium glutamicum which codes for pyruvate carboxylase (WO 99/18228),
the pps gene which codes for phosphoenol pyruvate synthase (Molecular and General Genetics 231 (2): 332-336 (1992)),
the ppc gene which codes for phosphoenol pyruvate carboxylase (WO 02/064808),
the pntA and pntB genes which code for transhydrogenase (European Journal of Biochemistry 158: 647-653 (1986)),
the rhtB gene which imparts homoserine resistance (EP-A-0 994 190),
the rhtC gene which imparts threonine resistance (EP-A-1 013 765),
the thrE gene of Corynebacterium glutamicum which codes for the threonine export protein (WO 01/92545),
the gdhA gene which codes for glutamate dehydrogenase (Nucleic Acids Research 11: 5257-5266 (1983); Gene 23: 199-209 (1983)),
the pgm gene which codes for phosphoglucomutase (WO 03/004598),
the fba gene which codes for fructose biphosphate aldolase (WO 03/004664),
the ptsH gene of the ptsHIcrr operon which codes for the phosphohistidine protein hexose phosphotransferase of the phosphotransferase system PTS (WO 03/004674),
the ptsI gene of the ptsHIcrr operon which codes for enzyme I of the phosphotransferase system PTS (WO 03/004674),
the crr gene of the ptsHIcrr operon which codes for the glucose-specific IIA component of the phosphotransferase system PTS (WO 03/004674),
the ptsG gene which codes for the glucose-specific IIBC component (WO 03/004670),
the lrp gene which codes for the regulator of the leucine regulon (WO 03/004665),
the fadR gene which codes for the regulator of the fad regulon (WO 03/038106),
the iclR gene which codes for the regulator of central intermediate metabolism (WO 03/038106),
the ahpC gene of the ahpCF operon which codes for the small sub-unit of alkyl hydroperoxide reductase (WO 03/004663),
the ahpF gene of the ahpCF operon which codes for the large sub-unit of alkyl hydroperoxide reductase (WO 03/004663),
the cysK gene which codes for cysteine synthase A (WO 03/006666),
the cysB gene which codes for the regulator of the cys regulon (WO 03/006666),
the cysJ gene of the cysJIH operon which codes for the flavoprotein of NADPH sulfite reductase (wO 03/006666),
the cysI gene of the cysJIH operon which codes for the haemoprotein of NADPH sulfite reductase (WO 03/006666),
the cysH gene of the cysJIH operon which codes for adenylyl sulfate reductase (WO 03/006666),
the rseA gene of the rseABC operon which codes for a membrane protein with anti-sigmaE activity (WO 03/008612),
the rseC gene of the rseABC operon which codes for a global regulator of the sigmaE factor (WO 03/008612),
the sucA gene of the sucABCD operon which codes for the decarboxylase sub-unit of 2-ketoglutarate dehydrogenase (WO 03/008614),
the sucB gene of the sucABCD operon which codes for the dihydrolipoyltranssuccinase E2 sub-unit of 2-ketoglutarate dehydrogenase (WO 03/008614),
the sucC gene of the sucABCD operon which codes for the β-sub-unit of succinyl-CoA synthetase (WO 03/008615),
the sucD gene of the sucABCD operon which codes for the α-sub-unit of succinyl-CoA synthetase (WO 03/008615),
the aceE gene which codes for the E1 component of the pyruvate dehydrogenase complex (WO 03/076635),
the aceF gene which codes for the E2 component of the pyruvate dehydrogenase complex (WO 03/076635), and
the rseB gene which codes the regulator of sigmaE factor activity (Molecular Microbiology 24 (2): 355-371 (1997)) can be enhanced.

It may furthermore be advantageous for the production of L-amino acids, in particular L-threonine, in addition to the enhancement of the eno gene, for one or more of the genes chosen from the group consisting of
the tdh gene which codes for threonine dehydrogenase (Journal of Bacteriology 169: 4716-4721 (1987)),
the mdh gene which codes for malate dehydrogenase (E.C. 1.1.1.37) (Archives in Microbiology 149: 36-42 (1987)),
the gene product of the open reading frame (orf) yjfA (Accession Number AAC77180 of the National Center for Biotechnology Information (NCBI, Bethesda, Md., USA), (WO 02/29080)),
the gene product of the open reading frame (orf) ytfP (Accession Number AAC77179 of the National Center for Biotechnology Information (NCBI, Bethesda, Md., USA), (WO 02/29080)),
the pckA gene which codes for the enzyme phosphoenol pyruvate carboxykinase (WO 02/29080),
the poxB gene which codes for pyruvate oxidase (WO 02/36797),
the dgsA gene which codes for the DgsA regulator of the phosphotransferase system (WO 02/081721) and is also known under the name of the mlc gene,
the fruR gene which codes for the fructose repressor (WO 02/081698) and is also known under the name of the cra gene,
the rpos gene which codes for the sigma³⁸ factor (WO 01/05939) and is also known under the name of the katF gene, and
the aspA gene which codes for aspartate ammonium lyase (WO 03/008603)
   to be attenuated, in particular eliminated or for the expression thereof to be reduced.

The term “attenuation” in this connection describes the reduction or elimination of the intracellular activity or concentration of one or more enzymes or proteins in a microorganism which are coded by the corresponding DNA, for example by using a weak promoter or a gene or allele which codes for a corresponding enzyme or protein with a low activity or inactivates the corresponding enzyme (protein) or gene and optionally combining these measures.

By attenuation measures, the activity or concentration of the corresponding protein is in general reduced to 0 to 75%, 0 to 50%, 0 to 25%, 0 to 10% or 0 to 5% of the activity or concentration of the wild-type protein or of the activity or concentration of the protein in the starting microorganism.

In addition to enhancement of the eno gene it may furthermore be advantageous for the production of L-amino acids, in particular L-threonine, to eliminate undesirable side reactions (Nakayama: “Breeding of Amino Acid Producing Microorganisms”, in: Overproduction of Microbial Products, Krumphanzl, Sikyta, Vanek (eds.), Academic Press, London, UK, 1982).

The microorganisms produced according to the invention can be cultured in the batch process (batch culture), the fed batch (feed process) or the repeated fed batch process (repetitive feed process). A summary of known culture methods is described in the textbook by Chmiel (Bioprozesstechnik 1. Einführung in die Bioverfahrenstechnik (Gustav Fischer Verlag, Stuttgart, 1991)) or in the textbook by Storhas (Bioreaktoren und periphere Einrichtungen (vieweg Verlag, Braunschweig/Wiesbaden, 1994)).

The culture medium to be used must meet the requirements of the particular strains in a suitable manner. Descriptions of culture media for various microorganisms are contained in the handbook “Manual of Methods for General Bacteriology” of the American Society for Bacteriology (Washington D.C., USA, 1981).

Sugars and carbohydrates, such as e.g. glucose, sucrose, lactose, fructose, maltose, molasses, starch and optionally cellulose, oils and fats, such as e.g. soya oil, sunflower oil, groundnut oil and coconut fat, fatty acids, such as e.g. palmitic acid, stearic acid and linoleic acid, alcohols, such as e.g. glycerol and ethanol, and organic acids, such as e.g. acetic acid, can be used as the source of carbon. These substances can be used individually or as a mixture.

Organic nitrogen-containing compounds, such as peptones, yeast extract, meat extract, malt extract, corn steep liquor, soya bean flour and urea, or inorganic compounds, such as ammonium sulfate, ammonium chloride, ammonium phosphate, ammonium carbonate and ammonium nitrate, can be used as the source of nitrogen. The sources of nitrogen can be used individually or as a mixture.

Phosphoric acid, potassium dihydrogen phosphate or dipotassium hydrogen phosphate or the corresponding sodium-containing salts can be used as the source of phosphorus. The culture medium must furthermore comprise salts of metals, such as e.g. magnesium sulfate or iron sulfate, which are necessary for growth. Finally, essential growth substances, such as amino acids and vitamins, can be employed in addition to the above-mentioned substances. Suitable precursors can moreover be added to the culture medium. The starting substances mentioned can be added to the culture in the form of a single batch, or can be fed in during the culture in a suitable manner.

The fermentation is in general carried out at a pH of 5.5 to 9.0, in particular 6.0 to 8.0. Basic compounds, such as sodium hydroxide, potassium hydroxide, ammonia or aqueous ammonia, or acid compounds, such as phosphoric acid or sulfuric acid, can be employed in a suitable manner to control the pH of the culture. Antifoams, such as e.g. fatty acid polyglycol esters, can be employed to control the development of foam. Suitable substances having a selective action, e.g. antibiotics, can be added to the medium to maintain the stability of plasmids. To maintain aerobic conditions, oxygen or oxygen-containing gas mixtures, such as e.g. air, are introduced into the culture. The temperature of the culture is usually 25° C. to 45° C., and preferably 30° C. to 40° C. Culturing is continued until a maximum of L-amino acids or L-threonine has formed. This target is usually reached within 10 hours to 160 hours.

The analysis of L-amino acids can be carried out by anion exchange chromatography with subsequent ninhydrin derivation, as described by Spackman et al. (Analytical Chemistry, 30: 1190-1206 (1958)) or it can be carried out by reversed phase HPLC, as described by Lindroth et al. (Analytical Chemistry 51: 1167-1174 (1979)).

The process according to the invention is used for the fermentative preparation of L-amino acids, such as, for example, L-threonine, L-isoleucine, L-valine, L-methionine, L-homoserine and L-lysine, in particular L-threonine.

The present invention is explained in more detail in the following with the aid of embodiment examples.

The minimal (M9) and complete media (LB) for Escherichia coli used are described by J. H. Miller (A Short Course in Bacterial Genetics (1992), Cold Spring Harbor Laboratory Press). The isolation of plasmid DNA from Escherichia coli and all techniques of restriction, ligation, Klenow and alkaline phosphatase treatment are carried out by the method of Sambrook et al. (Molecular Cloning—A Laboratory Manual (1989) Cold Spring Harbor Laboratory Press). Unless described otherwise, the transformation of Escherichia coli is carried out by the method of Chung et al. (Proceedings of the National Academy of Sciences of the United States of America 86: 2172-2175 (1989)).

The incubation temperature for the preparation of strains and transformants is 37° C.

### EXAMPLE 1

Construction of the Expression Plasmid pTrc99Aeno

The eno gene from E. coli K12 is amplified using the polymerase chain reaction (PCR) and synthetic oligonucleotides. Starting from the nucleotide sequence of the eno gene in E. coli K12 MG1655 (Accession Number AE000361, Blattner et al. (Science 277: 1453-1474 (1997)), PCR primers are synthesized (MWG Biotech, Ebersberg, Germany). The primers contain sequences for restriction enzymes which are marked by underlining in the nucleotide sequence shown below. The primer enol contains the restriction cleavage site for XabI, the primer eno2 contains that for HindIII.

eno1:

(SEQ ID No. 1)

5′ - GTTTGTCTAGAGTTTCAGTTTAACTAGTGAC - 3′
eno2:

(SEQ ID No. 2)

5′ - CCGGAGGCTGGCAAGCTTAAATCAG - 3′

The chromosomal E. coli K12 MG1655 DNA employed for the PCR is isolated according to the manufacturers instructions with “Qiagen Genomic-tips 100/G” (QIAGEN, Hilden, Germany). A DNA fragment approx. 1,381 bp in size can be amplified with the specific primers under standard PCR conditions (Innis et al. (1990) PCR Protocols. A Guide to Methods and Applications, Academic Press) with Vent-DNA polymerase (New England BioLabs, Frankfurt, Germany) (SEQ ID No. 3).

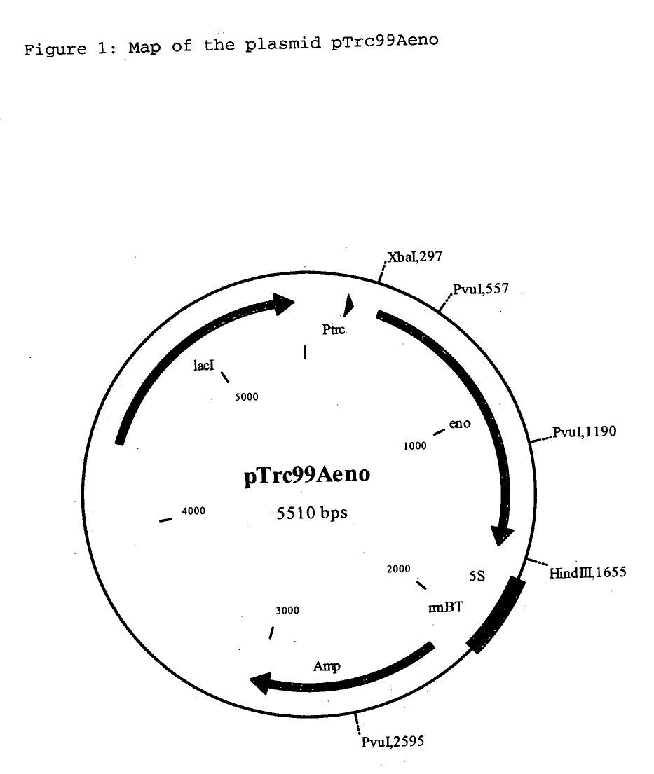

The amplified eno fragment is restricted with the restriction enzymes HindIII and XbaI and after purification (Purification Kit, QIAGEN, Hilden, Germany) checked in a 0.8% agarose gel. The vector pTrc99A (Pharmacia Biotech, Uppsala, Sweden) is cleaved with the enzymes HindIII and XbaI and ligation is carried out with the restricted eno fragment. The E. coli strain TOP10 One Shot (TOPO TA Cloning Kit, Invitrogen, Groningen, The Netherlands) is transformed with the ligation batch and plasmid-carrying cells are selected on LB agar, to which 50 μg/ml ampicillin is added. Successful cloning can be demonstrated after plasmid DNA isolation by control cleavage with the enzymes HindIII/XbaI and PvuI. The plasmid is called pTrc99Aeno (FIG. 1).

### EXAMPLE 2

Preparation of L-threonine with the Strain MG442/pTrc99Aeno

The L-threonine-producing E. coli strain MG442 is described in the patent specification U.S. Pat. No. 4,278,765 and deposited as CMIM B-1628 at the Russian National Collection for Industrial Microorganisms (VKPM, Moscow, Russia).

The strain MG442 is transformed with the expression plasmid pTrc99Aeno described in example 1 and with the vector pTrc99A and plasmid-carrying cells are selected on LB agar with 50 μg/ml ampicillin. Successful transformations can be demonstrated after plasmid DNA isolation by control cleavages with the enzymes HindIII/XbaI. The strains MG442/pTrc99Aeno and MG442/pTrc99A are formed in this manner. Selected individual colonies are then multiplied further on minimal medium with the following composition: 3.5 g/l Na₂HPO₄*2H₂₀, 1.5 g/l KH₂PO₄, 1 g/l NH₄Cl, 0.1 g/l. MgSO₄*7H₂O, 2 g/l glucose, 20 g/l agar, 50 mg/l ampicillin. The formation of L-threonine is checked in batch cultures of 10 ml contained in 100 ml conical flasks. For this, 10 ml of preculture medium of the following composition: 2 g/l yeast extract, 10 g/l (NH₄)₂SO₄, 1 g/l KH₂PO₄, 0.5 g/l MgSO₄*7H₂O, 15 g/l CaCO₃, 20 g/l glucose, 50 mg/l ampicillin are inoculated and the batch is incubated for 16 hours at 37° C. and 180 rpm on an ESR incubator from Kühner AG (Birsfelden, Switzerland). 250 μl portions of this preculture are transinoculated into 10 ml of production medium (25 g/l (NH4)₂SO₄, 2 g/1 KH₂PO₄, 1 g/l MgSO₄*7H₂O, 0.03 g/l FeSO₄*7H₂O, 0.018 g/l MnSO₄*1H₂O, 30 g/l CaCO₃, 20 g/l glucose, 50 mg/l ampicillin) and the batch is incubated for 48 hours at 37° C. For complete induction of the expression of the eno gene, 100 mg/l isopropyl β-D-thiogalactopyranoside (IPTG) are added in parallel batches. The formation of L-threonine by the starting strain MG442 is investigated in the same manner, but no addition of ampicillin to the medium takes place. After the incubation the optical density (OD) of the culture suspension is determined with an LP2W photometer from Dr. Lange (Dusseldorf, Germany) at a measurement wavelength of 660 nm.

The concentration of L-threonine formed is then determined in the sterile-filtered culture supernatant with an amino acid analyzer from Eppendorf-BioTronik (Hamburg, Germany) by ion exchange chromatography and post-column reaction with ninhydrin detection.

The result of the experiment is shown in Table 1.

TABLE 1

| Strain | Additives | OD (660 nm) | L-Threonine g/l |
|---|---|---|---|
| MG442 | — | 5.6 | 1.4 |
| MG442/pTrc99A | — | 3.8 | 1.3 |
| MG442/pTrc99Aeno | — | 2.6 | 1.7 |
| MG442/pTrc99Aeno | IPTG | 5.1 | 2.6 |

### BRIEF DESCRIPTION OF THE FIGURE

The length data are to be understood as approx. data. The abbreviations and designations used have the following meaning:

| | |
|---|---|
| Amp: | ampicillin resistance gene |
| lacI: | gene for the repressor protein of the trc |
| | promoter |
| Ptrc: | trc promoter region, IPTG-inducible |
| eno: | coding region of the eno gene |
| 5S: | 5S rRNA region |
| rrnBT: | rRNA terminator region |

The abbreviations for the restriction enzymes have the following meaning

| | |
|---|---|
| HindIII: | restriction endonuclease from Haemophilus |
| | influenzae R_{c} |
| PvuI: | restriction endonuclease from Proteus |
| | vulgaris |
| XbaI: | restriction endonuclease from Xanthomonas |
| | campestris |

1. Process for the preparation of L-amino acids, in particular L-threonine, wherein the following steps are carried out:
a) fermentation of the microorganisms of the Enterobacteriaceae family which produce the desired L-amino acid and in which the eno gene or nucleotide sequences or alleles which code for it are enhanced,
b) concentration of the desired L-amino acid in the medium or in the cells of the microorganisms, and
c) isolation of the desired L-amino acid, constituents of the fermentation broth and/or the biomass in its entirety or portions (≧0 to 100%) thereof optionally remaining in the product.

2. Process according to claim 1., wherein microorganisms in which further genes of the biosynthesis pathway of the desired L-amino acid are additionally enhanced are employed.

3. Process according to claim 1, wherein microorganisms in which the metabolic pathways which reduce the formation of the desired L-amino acid are at least partly eliminated are employed.

4. Process according to claim 1, wherein the expression of the polynucleotide(s) which code(s) for the eno gene is increased.

5. Process according to claim 1, wherein the regulatory and/or catalytic properties of the polypeptide (protein) for which the polynucleotide eno codes are improved or increased.

6. Process for the preparation of L-amino acids according to claim 1, wherein microorganisms of the Enterobacteriaceae family in which additionally at the same time one or more of the genes chosen from the group consisting of:
6.1 the thrABC operon which codes for aspartate kinase, homoserine dehydrogenase, homoserine kinase and threonine synthase,
6.2 the pyc gene which codes for pyruvate carboxylase,
6.3 the pps gene which codes for phosphoenol pyruvate synthase,
6.4 the ppc gene which codes for phosphoenol pyruvate carboxylase,
6.5 the pntA and pntB genes which code for transhydrogenase,
6.6 the rhtB gene which imparts homoserine resistance,
6.7 the rhtC gene which imparts threonine resistance,
6.8 the thrE gene which codes for the threonine export protein,
6.9 the gdhA gene which codes for glutamate dehydrogenase,
6.10 the pgm gene which codes for phosphoglucomutase,
6.11 the fba gene which codes for fructose biphosphate aldolase,
6.12 the ptsH gene which codes for the phosphohistidine protein hexose phosphotransferase,
6.13 the ptsI gene which codes for enzyme I of the phosphotransferase system,
6.14 the crr gene which codes for the glucose-specific IIA component,
6.15 the ptsG gene which codes for the glucose-specific IIBC component,
6.16 the lrp gene which codes for the regulator of the leucine regulon,
6.17 the fadR which codes for the regulator of the fad regulon,
6.18 the iclR gene which codes for the regulator of central intermediate metabolism,
6.19 the ahpC gene which codes for the small sub-unit of alkyl hydroperoxide reductase,
6.20 the ahpF gene which codes for the large sub-unit of alkyl hydroperoxide reductase,
6.21 the cysK gene which codes for cysteine synthase A,
6.22 the cysB gene which codes for the regulator of the cys regulon,
6.23 the cysJ gene which codes for the flavoprotein of NADPH sulfite reductase,
6.24 the cysI gene which codes for the haemoprotein of NADPH sulfite reductase,
6.25 the cysH gene which codes for adenylyl sulfate reductase,
6.26 the rseA gene which codes for a membrane protein with anti-sigmaE activity,
6.27 the rseC gene which codes for a global regulator of the sigmaE factor,
6.28 the sucA gene which codes for the decarboxylase sub-unit of 2-ketoglutarate dehydrogenase,
6.29 the sucB gene which codes for the dihydrolipoyltranssuccinase E2 sub-unit of 2-ketoglutarate dehydrogenase,
6.30 the sucC gene which codes for the β-sub-unit of succinyl-CoA synthetase,
6.31 the sucD gene which codes for the α-sub-unit of succinyl-CoA synthetase,
6.32 the aceE gene which codes for the E1 component of the pyruvate dehydrogenase complex,
6.33 the aceF gene which codes for the E2 component of the pyruvate dehydrogenase complex, and
6.34 the rseB gene which codes for the regulator of sigmaE factor activity
is or are enhanced are fermented.

7. Process for the preparation of L-amino acids according to claim 1, wherein microorganisms of the Enterobacteriaceae family in which additionally at the same time one or more of the genes chosen from the group consisting of:
7.1 the tdh gene which codes for threonine dehydrogenase,
7.2 the mdh gene which codes for malate dehydrogenase,
7.3 the gene product of the open reading frame (orf) yjfA,
7.4 the gene product of the open reading frame (orf) ytfp,
7.5 the pckA gene which codes for phosphoenol pyruvate carboxykinase,
7.6 the poxB gene which codes for pyruvate oxidase,
7.7 the dgsA gene which codes for the DgsA regulator of the phosphotransferase system,
7.8 the fruR gene which codes for the fructose repressor,
7.9 the rpoS gene which codes for the sigma³⁸ factor and
7.10 the aspA gene which codes for aspartate ammonium lyase
is or are attenuated, in particular eliminated or reduced in expression, are fermented.

8. Microorganisms of the Enterobacteriaceae family, in particular of the genus Escherichia, in which the eno gene or nucleotide sequences which code for it are present in enhanced form.

9. Microorganisms according to claim 8, wherein they produce L-threonine.
